(19) United States
(12) Patent Application Publication
(10) Pub. No.: US 2013/0224125 A1
(43) Pub. Date: Aug. 29, 2013

(54) ALCOHOL-FREE SLIGHTLY-ALCOHOLIC ORAL CARE COMPOSITION AND A PROCESS FOR PREPARING SAME

(71) Applicants: Narayanan S. Kolazi, Wayne, NJ (US); William E. Prosise, Ramsey, NJ (US); Robert J. Corring, Rockaway Twp, NJ (US)

(72) Inventors: Narayanan S. Kolazi, Wayne, NJ (US); William E. Prosise, Ramsey, NJ (US); Robert J. Corring, Rockaway Twp, NJ (US)

(21) Appl. No.: 13/813,567

(22) PCT Filed: Jul. 19, 2011
(86) PCT No.: PCT/US11/44484

§ 371 Date: May 3, 2013

Related U.S. Application Data
(60) Provisional application No. 61/370,300, filed on Aug. 3, 2010.

(51) Int. Cl. A61K 8/81; A61Q 11/00; A61K 8/46

(52) U.S. Cl. CPC A61K 8/8176; A61K 8/466; A61Q 11/00
USPC 424/52; 424/49; 424/57; 424/54; 424/55; 424/53

(57) ABSTRACT

An aqueous, heat and cold stable, non-alcoholic or slightly-alcoholic microemulsion based antimicrobial mouthwash composition with improved antimicrobial efficacy. The composition comprises a unique water-soluble matrix composite, at least one water-immiscible or water-insoluble antimicrobial agent, and optionally, a preservative or preservative system, a weak carboxylic acid, a coloring agent and other additives. Examples of antimicrobial agents include menthol, thymol, eucalyptol and/or methyl salicylate. A process for preparing the oral care composition is also disclosed.

### FIELD OF THE INVENTION

The present application relates to improved alcohol-free or slightly-alcoholic microemulsion based antimicrobial mouthwash compositions, and more particularly, an oral care mouthwash composition comprising a water-soluble matrix composite and a water-immiscible or water-insoluble antimicrobial agent and, optionally additives, and a process for preparing the same.

### BACKGROUND OF THE INVENTION

Mouthwashes or mouthrinses are liquid oral care preparations developed to clean and refresh the oral cavity or oral surface by inhibiting or killing the microorganisms that cause malodor, dental caries, tooth decay, gum diseases, gingivitis, and periodontal disorders. These types of compositions have been used for the last several decades in various compositions or formulations. However, the effectiveness of an antimicrobial mouthwash composition is based on its ability to deliver the active ingredient(s) contained therein, to kill the targeted microorganisms. This is predominantly so for antimicrobial mouthwash compositions wherein the exposure time of the targeted microorganism to the selected antimicrobial agent is usually of a short period.

The various types of conventional mouthwashes are used in different courses of therapy of oral cleanliness. These include (i) conventional mouthwash compositions which primarily sweeten the breath with one or more flavoring agents and are not structured to function in any noteworthy way to cleanse the oral cavity by preventing plaque formation therein; (ii) pre-rinse compositions, used immediately before brushing so as to make the plaque or calculus more amenable to elimination during subsequent brushing; (iii) gingivitis and/or tartar control rinses comprising antimicrobials such as phenols, sanguinaria, chlorhexidine and stannous fluoride; and antitartar or plaque fighters such as alkaline pyrophosphates and alkaline salts of benzoic acid. Some of these are disclosed in the review by K. S. Kornman, Dent. Placue Control Meas. Oral Hyg. Pratt. Proc., pp. 121-142 (1986).

Conventional mouthwashes have typically had relatively high levels of alcohol content, particularly, 10% to about 30% v/v of ethyl alcohol. The ethyl alcohol is employed as a disinfectant or solvent for the added excipients such as astringents, fluorides, colors, flavors, etc. Further, the higher quantity of alcohol is usually employed to provide a disinfection role since lower amounts are adequate to dissolve the various ingredients of the composition into solution. Ethyl alcohol also offers a preservative function for the mouthwash compositions during storage and is used to enhance organoleptic or aesthetic properties of flavor oil.

Moreover, reducing the quantity of ethyl alcohol in these types of mouthwash compositions has major disadvantages. Lowering the alcohol content results in decreased solubility of active ingredients and thereby lesser antimicrobial efficacy of the composition with regard to bad breath, plaque gum disease and the like. It is believed that the loss in antimicrobial efficacy is due to poor diffusion of selected antimicrobial agents and thereby reduced penetration into the plaque matrix. Hence, fairly high levels of ethyl alcohol have been employed to deliver desired antimicrobial kinetics, predominantly for compositions comprising essential or flavor oil as the active antimicrobial agents.

The use of alcohol has not been regarded as advantageous from an overall health perspective due to the following non-limiting reasons (i) a large number of children's and elderly cannot tolerate alcohol for health and safety related reasons; (ii) alcohol-containing preparations are often abused by alcoholics; (iii) alcoholic mouthwashes may irritate the protective layers of the mouth and throat, or dry out inflamed tissues.

Clearly, there is a substantial need for the development of a non-alcoholic mouthwash which has effective antimicrobial efficacy with respect to prevention or reduction of bad breath, the killing of oral bacteria or elimination or reduction of plaque. Hence, various attempts have been made at developing non-alcoholic mouthwashes. For example, U.S. Pat. No. 4,919,918 to Cole et al. and U.S. Pat. No. 4,971,785 to Wilson et al. disclose dry compositions which are dissolved in water immediately prior to use.

U.S. Pat. Nos. 5,292,527 and 5,407,664 to Bausch & Lomb Incorporated reveals a non-alcoholic, aqueous mouthwash composition comprising a dispersion system that consists of a non-ionic surfactant, hydrogenated castor oils and a polyoxyethylene polyoxypropylene block copolymer having about 50% to about 90% ethylene oxide, a humectant and a cationic antimicrobial agent such as cetylpyridium chloride. The composition allegedly exhibits a homogeneous, uniform appearance and high degree of bactericidal efficacy.

U.S. Pat. No. 5,560,906 to Oral Technology Laboratories, Inc. discloses a pleasant tasting antimicrobial mouthwash formulation which maintains clarity for removal of dental plaque where no alcohol, sugar, artificial sweeteners are used making it suitable for safe use by alcoholics, diabetics, persons under medical treatment or taking medications which preclude the use of alcohol, hospitalized patients, prison inmates, minors and all other persons who cannot or should not subject themselves to alcohol, sugar, or artificial sweeteners. The formulation consists of water, glycerin, sodium benzoate, cetylpyridinium chloride, citric acid, malted, xylitol, a flavoring agent to give a pleasant though biting taste, and a coloring agent.

U.S. Pat. No. 5,817,295 to Warner and Lambert Company describes a substantially alcohol-free mouthwash which provides effective breath freshening and antimicrobial oral hygiene for everyday use. The mouthwash composition is comprised of a unique mix of non-ionic and ionic surfactants, essential oils, and flavor oils such as thymol, methyl salicylate, menthol, eucalyptol and other excipients that provide the benefits of an alcohol-based composition without the inherent drawbacks.

U.S. Pat. No. 5,707,610 to Den-Mat Corporation discloses an antibacterial oral hygiene composition in aqueous form comprising (a) sodium benzoate; (b) a weak carboxylic acid; (c) buffering agent capable of buffering the composition to a pH of about 3.0 to about 8.0; (d) surfactant; (e) sodium saccharin; (f) flavoring agent; and (g) sufficient water to total 100%.

In view of forgoing facts, clearly, there remains a need for a stable, non-alcoholic or slightly-alcoholic antimicrobial mouthwash composition which is capable of providing improved or desired antimicrobial efficacy preferably in shorter exposure duration on oral cavity. The present application describes antimicrobial mouthwash compositions comprising a water-soluble matrix composite and a water-insoluble/immiscible antimicrobial agent.

### SUMMARY OF THE INVENTION

In accordance with certain aspects, the present application provides an aqueous, stable, non-alcoholic or slightly-alcoholic antibacterial mouthwash composition with an improved antimicrobial efficacy, wherein said composition comprises (i) a water-soluble matrix composite made of (a) a water-soluble polymer, and (b) a water-soluble surfactant; (ii) at least one water-immiscible or water-insoluble antibacterial agent; and (iii) optionally, a preservative or preservative system, a weak carboxylic acid, a coloring agent and other additives. The water-soluble matrix disclosed herein comprises a complex of (a) and (b) having a lower critical micelle concentration (cmc) than a composition without the water-soluble polymer.

The present application also describes a heat and cold stable mouthwash composition wherein the composition is stable for at least six months at room temperature or stable for at least 3 freeze/thaw cycles wherein temperature cycled from 50° C. to −24° C. in every 24 hours or stable for at least 2 weeks at about 50° C.

In accordance with another aspect, the present application describes an antibacterial mouthwash composition which is capable of inhibiting (static) or killing (cidal) gram (−) and/or gram (+) bacterial strains and fungal strains.

In accordance with certain embodiments, the composition includes a water-immiscible or water-insoluble antibacterial agent selected from the group consisting of essential oils, synthetic antimicrobial agents, antimicrobial agents isolated from essential oils and their semi-synthetic derivatives, plant extracts or their bioactive fractions, naturally available disinfecting plant products alone or in combination.

In accordance with one aspect, there is provided a process for preparing a aqueous, stable, non-alcoholic or slightly-alcoholic antibacterial mouthwash composition with an improved antimicrobial efficacy comprising the steps of (i) preparing a mixture of (a) a water-soluble matrix composite and (b) an essential oil based antibacterial agent; (ii) dissolving the mixture of step (i) in an aqueous medium, optionally comprising an effective amount of alcohol to yield a clear microemulsion; and (iii) incorporating preservative, weak carboxylic acid, coloring agent and additives, if any, to the microemulsion of step (ii) and vigorously mixing the composition to result in stable, homogenous, optically clear mouthwash composition.

The antibacterial mouthwash composition described herein can be formulated in various other deliverable forms including, but not limited to, oral disinfecting liquids, oral disinfecting solids, oral gargling compositions, solid dosage forms, controlled release forms, and/or sustained release forms.

The additives for preparing the desired antibacterial mouthwash composition may be selected from flavors, thickeners, defoamers, buffers, sweeteners, humectants, softeners and/or astringents, etc.

### DETAILED DESCRIPTION OF THE INVENTION

While this specification concludes with claims particularly pointing out and distinctly claiming that, which is regarded as the invention, it is anticipated that the invention can be more readily understood through reading the following detailed description of the invention and study of the included examples.

By the term “comprising” herein is meant that various optional, compatible components can be used in the compositions herein, provided that the important ingredients are present in the suitable form and concentrations. The term “comprising” thus encompasses and includes the more restrictive terms “consisting of” and “consisting essentially of” which can be used to characterize the essential ingredients, water, water-soluble polymer, water-soluble surfactant, water-immiscible or water-insoluble antibacterial agent, preservative, carboxylic acid, coloring agent and additives if any of the present alcohol-free oral care mouth wash compositions.

All percentages, parts, proportions and ratios as used herein, are by weight of the total composition, unless otherwise specified. All such weights as they pertain to listed ingredients are based on the active level and, therefore; do not typically include solvents or by-products that may be included in commercially available materials, unless otherwise specified.

All references to singular characteristics or limitations of the present application shall include the corresponding plural characteristic or limitation, and vice-versa, unless otherwise specified or clearly implied to the contrary by the context in which the reference is made.

Numerical ranges as used herein are intended to include every number and subset of numbers contained within that range, whether specifically disclosed or not. Further, these numerical ranges should be construed as providing support for a claim directed to any number or subset of numbers in that range.

The term “about” can indicate a variation of 10 percent of the value specified; for example about 50 percent carries a variation from 45 to 55 percent. For integer ranges, the term about can include one or two integers greater than and less than a recited integer.

The patents and publications referred to herein are hereby incorporated by reference to the extent necessary to understand the present invention.

As used herein, the words “preferred,” “preferably” and variants refer to embodiments of the application that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the application.

References herein to “one embodiment,” “one aspect” or “one version” or “one objective” of the application include one or more such embodiment, aspect, version or objective, unless the context clearly dictates otherwise.

The term “oral care composition” or “mouthwash composition” as used herein means a composition that provides either a cosmetic, prophylactic or therapeutic benefit within the oral cavity of the user. Further, the term “oral care composition” or “mouth wash composition” refers to a product which is not intentionally swallowed for the purposes of administration of therapeutic agents, but is retained in the oral cavity for a sufficient period of time to contact substantially all of the tooth surfaces and/or mucosal tissues for purposes of oral activity. The term “tooth surfaces” includes pits, fissures, occlusal surfaces, cleft, crevices, grooves, depressions, interstices, irregularities, inter-proximal surfaces between the teeth and/or along the gum line, the smooth surfaces of teeth, and/or the grinding or biting surfaces of a tooth.

The “oral care composition” or “mouth wash composition” described herein may be used to treat or cure the disease conditions of oral cavity pertaining to caries, plaque, breath malodor, gingivitis, and other periodontal related disorders.

As used herein, the term “polymer” is meant to encompass oligomer, and includes, without limitation, homopolymers, copolymers, terpolymers, etc. The polymers described herein can also be linear, branched and/or crosslinked polymers.

As used herein, the term “water-soluble,” when used in relation to polymers and polymer complexes, refers to polymers and polymer complexes that form a solution in water that is free of insoluble polymer particles. The determination that a solution is free of insoluble polymer particles can be made using conventional light scattering techniques or by passing the solution through a sufficiently fine filter screen capable of capturing insoluble polymer particles. As a non-limiting example, an aqueous solution containing 5 percent by weight of a polymer can be prepared and poured through a U.S. Standard Sieve No. 100 (150μ), and no particles are left on the screen. Alternatively, the turbidity of an aqueous solution containing 2.5 percent by weight of a polymer at a pH of from 5-9, may be measured using a turbidimeter or nephelometer. A reading of less than 20 nephelometric turbidity units (NTU) indicates the water-solubility of the polymer or polymer complex.

As used herein, the term “antibacterial agent” or “antimicrobial agent” or “hydrophobic agent” refers to a therapeutic or prophylactic agent such as essential oils, synthetic antimicrobial agents, antimicrobial agents isolated from essential oils and their semi-synthetic derivatives, plant extracts or their bioactive fractions, naturally available disinfecting plant products alone or in combination.

Disclosed herein is an aqueous, stable, non-alcoholic or slightly-alcoholic antimicrobial mouthwash composition with an improved antimicrobial efficacy, wherein said composition comprises (i) a water-soluble matrix composite made of (a) a water-soluble polymer, and (b) a water-soluble surfactant; (ii) at least one water-immiscible or water-insoluble antibacterial agent; and (iii) optionally, a preservative or preservative system, a weak carboxylic acid, a coloring agent and other additives. The water-soluble matrix disclosed herein comprises a complex of (a) and (b) having lower critical micelle concentration (cmc) than a composition without the water-soluble polymer.

In accordance with particular embodiments, the compositions include a water soluble matrix of a water soluble polymer and a water soluble surfactant. The matrix is in the form of a complex that stabilizes the hydrophobic water-immiscible or water-insoluble antibacterial or antimicrobial agent present in the composition as a micro-emulsion wherein the hydrophobic agent is present as particles in the micro/nanoparticle range. In accordance with certain embodiments of the present application, the compositions exhibit visual clarity and can be diluted to form use compositions of various concentrations of the hydrophobic agent or material. The term “complex” is used broadly to refer to a surfactant-polymer combination wherein the surfactant and polymer interact to provide a lower surface tension than either one of the components alone. Although not wishing to be bound by theory, it is theorized that the polymer-surfactant complex functions to stabilize the hydrophobic material in the composition. The stable nano or microemulsion can provide a solvent-free or reduced solvent system for delivering the antimicrobial hydrophobic agents.

Aqueous compositions containing a hydrophobic material, and, more particularly, a water soluble matrix of a water soluble polymer and a water soluble surfactant in the form of a complex are described in U.S. patent application Ser. No. 11/496,599, filed Jul. 31, 2006, U.S. patent application Ser. No. 11/803,108, filed May 11, 2007, and International Publication No. WO2008/016837, filed Jul. 27, 2007, the contents of which are hereby incorporated by reference.

The antimicrobial mouthwash described herein includes an aqueous based delivery system for water-insoluble or water-immiscible antimicrobial agents. The delivery system includes a water soluble matrix of a polymer and a surfactant. The water soluble matrix may be in the form of a complex. The delivery system in accordance with this aspect of the application includes a water soluble nanoparticulate dispersion/emulsion of selected hydrophobic antimicrobial agents in the defined water soluble matrix.

The water-insoluble or water-immiscible hydrophobic agents that are useful herein are not particularly limited. Hydrophobic agents are substantially insoluble in water. By the term “substantially insoluble,” it is meant that for all practical purposes, the solubility of the compound in water is insufficient to make the compound practicably usable without some modification either to increase its solubility or dispersability in water, so as to increase the compound's bioavailability or avoid the use of excessively large volumes of solvent. Substantially water insoluble materials usually include those having a solubility of less than 1 grain per liter of water at room temperature conditions.

The water-insoluble or water-immiscible antimicrobial agents for preparing an antimicrobial mouthwash composition may be selected from the group including, but not limited to, essential oils such as peppermint oil, spearmint oil, other mint oils, clove oil, wintergreen oil, anise oil, tea tree oil, lavender oil, pine oil, lemongrass oil, lemon oil, parsley oil, orange oil, clove oil, thyme oil, grapefruit oil, clove bud oil, aniseed oil, basil oil, black pepper oil, camphor oil, cananga oil, cardamom oil, cassia oil, cedarwood oil, cinnamon bark oil, cinnamon leaf oil, citrus oil, mint-type oils, citronella oil, eucalyptus oil, fennel oil, geranium oil, ginger oil, guaiacwood oil, juniper berry oil, lime oil distilled, litsea cubeba oil, patchouli oil, berry oil, pimento leaf oil, sandalwood oil, sage oil, sassafras oil, vetyver oil, bergamot oils, spice oils, origanum oil, pimento oil, buchu oil, caraway oil, carrot seed oil, copaiba oil, geranium oil, rosemary oil, targette oil, mace oil, nutmeg oil, cypress oil, cinnamon oil, coconut oil, fish oil, palm oil, mineral oil, apricot oil, cassia oil, castor oil, coriander oil, corn oil, cottonseed oil, peanut oil, soyabean oil, vegetable oil, pine seed oil, abyssinica oil, macadamia nut oil, limnanthes alba oil alone or in combination.

Further, the isolated water-insoluble or water-immiscible constituents of essential oils or their semi-synthetic derivatives include, but are not limited to, menthone, menthyl acetate, neomenthol, piperitone, pulegone, betacaryophyllene, betacaryophyllene-epoxide, alpha-pinene, beta-pinene, germacrene-D, 1,8-cineol, linalool, menthofurane, camphene, beta-hexenyl phenylacetate, d-limonene, 1-limonene, d1-limonene, alpha-citral, beta-citral (geranol), alpha-terpinene, gamma-terpinene, 2-dodecanal, 2-pentenal, cadiene, decylaldehyde, linalool, terpineol, linalyl esters, terpinyl acetate, decanal, C₈ to C₁₀₋₁₂ aldehydes, acids, amyl salicylate, cavacrol, dihydroeugenol, eugenol, hexyl eugenol, hexyl salicylate, isoeugenol, methyl eugenol, methyl isoeugenol, methyl salicylate, tert-butyl cresol, thymol, vanillin, cedrene, cineole, citral, citronellal, citronellol, cymene, paradihydrolinalool, dihydromyrcenol (DH myrcenol), farnesol, hexyl cinnamaldehyde, hydroxycitronallol, hydroxycitronellal, isocitral, linalool, longifolene, menthol, nerol, nerolidiol, phellendrene, terpinene, tetrahydromyrcenol (TH myrcenol), carvacrol, dihydroguaiaretic acid, nerolidole, gamma-decalactone and delta-decalactone, monocaprin, monolaurin, cinnamic acid, decanoic acid, 3-hydroxydecanoic acid, 9-decenoic acid, senecionic acid, nonanol, decanol, nonanal, decanal, amyl propionate, anethole, anisic aldehyde, cis-3-hexenol, damascone, ethyl acetoacetate, isoamyl acetate, menthol laevo, methyl cinnamate, cyclamen aldehyde, diphenyl oxide, ethyl vanilin, eucalyptol, L-methyl acetate, longifolene, menthol crystals, methyl cedryl ketone, methyl chavicol, methyl salicylate, musk ambrette, musk ketone, musk xylol, phenyl ethyl alcohol, vanilin, I-carvone, terpenes, alpha-citronellol, citronellyl acetate, citronellyl nitrile, para-cymene, dihydroanethole, dihydrocarveol, d-dihydrocarvone, dihydrolinalool, dihydromyrcene, dihydromyrcenol, dihydromyrcenyl acetate, dihydroterpineol, dimethyloctanal, dimethyloctanol, dimethyloctanyl acetate, estragole, ethyl-2 methylbutyrate, fenchol, geraniol, geranyl acetate, geranyl nitrile, hexenal, trans-2-hexenol, cis-3-hexenyl isovalerate, cis-3-hexanyl-2-methylbutyrate, hexyl isovalerate, hexyl-2-methylbutyrate, hydroxycitronellal, lonone, isobornyl methylether, linalool oxide, linalyl acetate, menthane hydroperoxide, I-methyl acetate, methyl hexyl ether, methyl-2-methylbutyrate, 2-methylbutyl isovalerate, myrcene, nerol, neryl acetate, 3-octyl acetate, phenyl-ethyl-2-methylbutyrate, cis-pinane, pinane hydroperoxide, pinanol, pine ester, alpha-pinene oxide, plinol, plinyl acetate, pseudo lonone, rhodinol, rhodinyl acetate, alpha-terpinene, gamma-terpinene, terpinene-4-ol, terpinolene, terpinyl acetate, tetrahydrolinalool, tetrahydrolinalyl acetate, phellandrene, pinene, methylheptenone, safrol, eugenyl acetate, caryophyllene, borneol, bornyl esters, camphor, menthyl esters, safrole, acetaldehyde, chavicol, cinnamyl acetate, α-thujone, β-thujone, fenchone, naturally available disinfecting plant products/extracts like neem, turmeric, cloves, alone or in combination.

Other suitable water-soluble, water-miscible, water-insoluble or water-immiscible antimicrobial compounds can also be included in the preparation of antimicrobial mouthwash compositions. Examples include, but are not limited to, Halogenated Diphenyl Ethers: 2′,4,4′-trichloro-2′-hydroxy-diphenyl ether (Triclosan) 2,2′-dihydroxy-5,5′-dibromo-diphenyl ether. Halogenated Salicylanilides: 4′,5-dibromosalicylanilide 3,4′,5-trichlorosalcylanilide, 3,4′,5-tribromosalicylanilide, 2,3,3′,5-tetrachlorosalicylanilide, 3,3,3′,5-tetrachlorosalicylanilide, dibromo-3′-trifluoromethyl salicylanilide, 5-n-octanoyl-3′-trifluoromethyl salicylanilide, 3,5-dibromc-4′-trifluoromethyl salicylanilide, 3,5-dibromo-3′-trifluoro methyl salicylanilide (Fluorophene). Benzoic Esters: Methyl-p-Hydroxybenzoic Ester, Ethyl-p-Hydroxybenzoic Ester, Propyl-p-Hydroxybenzoic Ester, Butyl-p-Hydroxybenzoic Ester. Halogenated Carbanilides: 3,4,4′-trichlorocarbanilide, 3-trifluoromethyl-4,4′-dichlorocarbanilide, 3,3,4′-trichlorocarbanilide. Phenolic Compounds (including phenol and its homologs, mono- and poly-alkyl and aromatic halo (e.g. F, Cl, Br, I)— phenols, resorcinol and catechol and their derivatives and bisphenolic compounds). Such compounds include inter alia: Phenol and its Homologs: Methyl-Phenol, Methyl-Phenol, Methyl-Phenol, Ethyl-Phenol, 2,4-Dimethyl-Phenol, 2,5-Dimethyl-Phenol, 3,4-Dimethyl-Phenol, 2,6-Dimethyl-Phenol, 4-n Propyl-Phenol, 4-n-Butyl-Phenol, 4-n-Amyl-Phenol, 4-tert-Amyl-Phenol, 4-n-Hexyl-Phenol, 4-n-Heptyl-Phenol, 2-Methoxy-4-(2-Propenyl)-Phenol, 2-Isopropyl-5-Methyl-Phenol. Mono- and Poly-Alkyl and Aralkyl Halophenols: Methyl-p-Chlorophenol, Ethyl-p-Chlorphenol, n-Propyl-p-Chlorophenol, n-Butyl-p-Chlorophenol, n-Amyl-p-Chlorophenol, sec-Amyl-p-Chlorophenol, n-Hexyl-p-Chloraphenol, Cyclohexyl-p-Chlorophenol, n-Heptyl-p-Chlorophenol, n-Octyl-p-Chloraphenol, O-Chlorophenol, Methyl-o-Chlorophenol, Ethyl-o-Chlorophenol, n-Propyl-o-Chloraphenol, n-Butyl-o-Chlorophenol, n-Amyl-o-Chlorophenol, tert-Amyl-o-Chlorophenol, n-Hexyl-o-Chlarophenol, n-Heptyl-o-Chloropenol, p-Chlorophenol, o-Benzyl-p-Chlorophenol, o-Benzyl-m-methyl-p-Chlorophenol, o-Benzyl-m, m-dimethyl-p-Chlorophenol, o-Phenylethyl-p-Chlorophenol, o-Phenylethyl-m-methyl-p-Chlorophenol, 3-Methyl-p-Chlorophenol, 3,5-Dimethyl-p-Chlorophenol, 6-Ethyl-3-methyl-p-Chlorophenol, 6-n-Propyl-3-methyl-p-Chlorophenol, 6-iso-propyl-3-methyl-p-Chlorophenol, 2-Ethyl-3,5-dimethyl-p-Chlorophenol, 6-sec-Butyl-3-methyl-p-Chlorophenol, 2-iso-Propyl-3,5-dimethyl-p-Chlorophenol, 6-Diethylmethyl-3-methyl-p-Chlorophenol, 6-iso-Propyl-2-ethyl-3-methyl-p-Chlorophenol, 2-sec Amyl-3,5-dimethyl-p-Chlorophenol, 2-Diethylmethyl-3,5-dimethyl-p-Chlorophenol, 6-sec Octyl-3-methyl-p-Chlorophenol, p-Bromophenol, Methyl-p-Bromophenol, Ethyl-p-Bromophenol, n-Propyl-p-Bromophenol, n-Butyl-p-Bromophenol, n-Amyl-p-Bromophenol, sec-Amyl-p-Bromophenol, n-Hexyl-p-Bromophenol, cyclohexyl-p-Bromophenol, o-Bromophenol, tert-Amyl-o-Bromophenol, n-Hexyl-o-Bromophenol, n-Propyl-m,m-Dimethyl-o-Bromophenol, 2-Phenyl Phenol 4-Chlora-2-methyl phenol, 4-chloro-3-methyl phenol, 4-chloro-3,5-dimethyl phenol, 2,4-dichloro-3,5-dimethyl phenol, 3,4,5,6-tetrabromo-2-methylphenol, 5-methyl-2-pentylphenol, 4-isopropyl-3-methylphenol, 5-chloro-2-hydroxydiphenyl methane. Resorcinol and Its Derivatives: Resorcinol Methyl-Resorcinol, Ethyl-Resorcinol, n-Propyl-Resorcinol, n-Butyl-Resorcinol, n-Amyl-Resorcinol, n-Hexyl-Resorcinol, n-Heptyl-Resorcinol, n-Octyl-Resorcinol, n-Nonyl-Resorcinol, Phenyl-Resorcinol, Benzyl-Resorcinol, Phenylethyl-Resorcinol, Phenylpropyl-Resorcinol, p-Chlorobenzyl-Resorcinol, 5-Chloro-2,4-Dihydroxydiphenyl Methane, 4′-Chloro-2,4-Dihydroxydiphenyl Methane, 5-Bromo-2,4-Dihydroxydiphenyl Methane, 4″-Bromo-2,4-Dihydroxydiphenyl Methane. Bisphenolic Compounds: Bisphenol A, 2′-methylene bis(4-chlorophenol), 2,2′-methylene bis(3,4,6-trichlorophenol) (hexachlorophene), 2,2′-methylene bis(4-chloro-6-bromophenol), bis-(2-hydroxy-3,5-dichlorophenyl)sulfide, bis-(2-hydroxy-5-chlorobenzyl)sulfide. Other exemplary antimicrobial agents include cetyl pyridium chloride, domiphen bromide, quaternay ammonium salts, zinc compounds, sanguinanine soluble pyrophosphates, fluorides, alexidine, octonidine, EDTA, chlorhexidine is chlorhexidine gluconate, benzoic acid, formaldehyde, potassium chlorate, tyrothricin, gramicidin, iodine, sodium perborate, and urea peroxide.

The antimicrobial agents present in the aqueous or solid composition at a wide range of concentrations depending on the antimicrobial agent and the use of the composition. For mouthwash concentrates, the hydrophobic material will typically be present in an amount by weight of about 1% to about 40%, more particularly from about 1.5% to about 30% and in accordance with certain embodiments from about 2% to about 20% of the concentrate. For use compositions, the hydrophobic agent will typically be present in an amount by weight of about 1 ppm to about 10000 ppm, more particularly from about 2 ppm to about 5000 ppm and in accordance with certain embodiments from about 5 ppm to about 4000 ppm of the diluted mouthwash oral care use composition. The antimicrobial agent may be present in an amount of about 10 ppm to about 40% by weight of the composition and more particularly from about 10 ppm to about 20% wt of the composition.

The hydrophobic agent is present in the composition as an emulsion. The particle size of the hydrophobic agent in the composition typically falls within the range of from about 5 to 1000 nm, more particularly from about 5 to 500 nm, still more particularly from about 10 to 100 nm and in accordance with certain embodiments from about 10 to 30 nm. Particle size refers to average particle radius and can be determined using dynamic light scattering techniques and equipment known to those of skill in the art. The compositions in accordance with certain aspects of the invention are visually clear due primarily to the small particle size of the hydrophobic material. Optical clarity can be measured using a turbidimeter or nephelometer. A reading of less than 200 nephelometric turbidity units (NTU), more particularly less than about 100 NTU at 25° C. typically indicates that the hydrophobic material is stable in the solution.

Water soluble polymers useful in the present application include those capable of forming a complex with a water soluble surfactant wherein the complex facilitates formation of a micro/nanoemulsion or micro/nanodispersion of the water-insoluble or water-immiscible hydrophobic active ingredient in the composition. Examples of typical polymer species include, but are not limited to, Lactam/Pyrrolidone based polymers, Polyvinyl pyrrolidone/polyvinyl caprolactam, Pyrrolidone co-polymers, Vinyl acetate-Vinylpyrrolidone co-polymers, Alkylated graft Vinylpyrrolidone co-polymers, Dimethylaminoethylmethacrylate Vinylpyrrolidone co-polymers, Acrylic acid/ester/salt-Vinylpyrrolidone co-polymers, Vinylpyrrolidone/Vinyl caprolactam co-polymers, Alpha olefin maleic acid/ester co-polymers, Styrene maleic acid co-polymers, Alkyl vinyl ether-maleic acid/ester/salts co-polymers, Alpha olefin Polymers: Polyacrylates/polyvinyl derivatives, Poly alkylacrylate/alkylacrylic esters/amides/salts, Polyvinyl alcohol/acetates, Natural polymers, Cellulosic derivatives, Modified Starch and/or alginates.

The water soluble polymer typically is used in an amount sufficient to form a complex with the surfactant and interact with the surfactant to lower the cmc of the system as compared to a system without the polymer. For certain embodiments, the water soluble polymer will be present in an amount by weight percent of about 0.1% to about 40%, more particularly from about 0.15% to about 30% and in accordance with certain embodiments from about 0.2% to about 20% of the concentrate. For use compositions, the water soluble polymer will typically be present in an amount by weight of about 1 ppm to about 10,000 ppm, more particularly from about 2 ppm to about 5,000 ppm and in accordance with certain embodiments from about 5 ppm to about 4,000 ppm of the diluted use composition.

The suitable surfactant for preparing antimicrobial mouthwash composition may be selected from anionic, non-ionic, amphoteric, cationic and mixtures thereof. The following types of surfactants are representative of the surfactants that can be used:

### (a) Anionic Surfactants:

Anionic surfactants are particularly useful in accordance with certain embodiments of the present application. Surfactants of the anionic type that may be useful include:

1. Sulfonates and Sulfates: Suitable anionic surfactants include sulfonates and sulfates such as alkyl sulfates, alkylether sulfates, alkyl sulfonates, alkylether sulfonates, alkylbenzene sulfonates, alkylbenzene ether sulfates, alkylsulfoacetates, secondary alkane sulfonates, secondary alkylsulfates, alkyl sulfosuccinates and the like. Further, examples of anionic surfactants include water-soluble salts of higher fatty acid monoglyceride monosulfates, such as the sodium salt of the monosulfated monoglyceride of hydrogenated coconut oil fatty acids, higher alkyl sulfates such as sodium lauryl sulfate, alkyl aryl sulfonates such as sodium dodecyl benzene sulfonate, higher alkyl sulfoacetates, higher fatty acid esters of 1,2-dihydroxy propane sulfonate, and the substantially saturated higher aliphatic acyl amides of lower aliphatic amino carboxylic acid compounds, such as those having 12 to 16 carbons in the fatty acid, alkyl or acyl radicals, and the like. Further, the use of these sarcosinate compounds in the oral care compositions of the present application is particularly advantageous since these materials exhibit a prolonged and marked effect in the inhibition of acid formation in the oral cavity due to carbohydrate breakdown in addition to exerting some reduction in the solubility of tooth enamel in acid solutions.
2. Examples include, but are not limited to: alkyl ether sulfonates such as lauryl ether sulfates such as POLYSTEP B 12 (n=3 4, M=sodium) and B22 (n=12, M=ammonium) available from Stepan Company, Northfield, 111. and sodium methyl taurate (available under the trade designation NIKKOL CMT30 from Nikko Chemicals Co., Tokyo, Japan); secondary alkane sulfonates such as Hostapur SAS which is a Sodium (C 14 C17) secondary alkane sulfonates (alpha-olefin sulfonates) available from Clariant Corp., Charlotte, N.C.; methyl-2-sulfoalkyl esters such as sodium methyl-2-sulfo(C12 16)ester and disodium 2-sulfo(C12 C16)fatty acid available from Stepan Company under the trade designation ALPHASTE PC-48; alkylsulfoacetates and alkylsulfosuccinates available as sodium laurylsulfoacetate (under the trade designation LANTHANOL LAL) and disodiumlaurethsulfosuccinate (STEPANMILD SL3), both from Stepan Company; alkylsulfates such as ammoniumlauryl sulfate commercially available under the trade designation STEPANOL AM from Stepan Company.
3. Phosphates and Phosponates: Suitable anionic surfactants also include phosphates such as alkyl phosphates, alkylether phosphates, aralkylphosphates, and aralkylether phosphates. Examples include a mixture of mono-, di- and tri-(alkyltetraglycolether)-o-phosphoric acid esters generally referred to as trilaureth-4-phosphate commercially available under the trade designation HOSTAPHAT 340KL from Clariant Corp., as well as PPG-5 ceteth 10 phosphate available under the trade designation CRODAPHOS SG from Croda Inc., Parsipanny, N.J.
4. Amine Oxides: Suitable anionic surfactants also include amine oxides. Examples of amine oxide surfactants include those commercially available under the trade designations AMMONYX LO, LMDO, and CO, which are lauryldimethylamine oxide, laurylamidopropyldimethylamine oxide, and cetyl amine oxide, all from Stepan Company.

### (b) Amphoteric Surfactants:

Surfactants of the amphoteric type include surfactants having tertiary amine groups which may be protonated as well as quaternary amine containing zwitterionic surfactants. Those that may be useful include:

1. Ammonium Carboxylate Amphoterics: Examples of such amphoteric surfactants include, but are not limited to: certain betaines such as cocobetaine and cocamidopropyl betaine (commercially available under the trade designations MACKAM CB-35 and MACKAM L from McIntyre Group Ltd., University Park, III); monoacetates such as sodium lauroamphoacetate; diacetates such as disodium lauroamphoacetate; amino- and alkylamino-propionates such as lauraminopropionic acid (commercially available under the trade designations MACKAM IL, MACKAM 2L, and MACKAM 151L, respectively, from McIntyre Group Ltd.).
2. Ammonium Sulfonate Amphoterics: This class of amphoteric surfactants are often referred to as “sultaines” or “sulfobetaines”. Examples include cocamidopropylhydroxysultaine (commercially available as MACKAM 50-SB from McIntyre Group Ltd.).

### (c) Nonionic Surfactants:

Surfactants of the nonionic type that may be particularly useful include:

### 1. Polyethylene Oxide Extended Sorbitan Monoalkylates (i.e., Polysorbates).

### 2. Polyalkoxylated Alkanols.

Surfactants such as those commercially available under the trade designation BRIJ from ICI Specialty Chemicals, Wilmington, Del. having an HLB of at least about 14 may be useful.

### 3. Polyalkoxylated Alkylphenols.

Examples of surfactants of this type include polyethoxylated octyl or nonyl phenols having HLB values of at least about 14, which are commercially available under the trade designations ICONOL and TRITON, from BASF Corp., Performance Chemicals Div., Mt. Olive, N.J. and Union Carbide Corp., Danbury, Conn., respectively. Examples include TRITON X100 (an octyl phenol having 15 moles of ethylene oxide available from Union Carbide Corp., Danbury, Conn.) and ICONOL NP70 and NP40 (nonyl phenol having 40 and 70 moles of ethylene oxide units, respectively, available from BASF Corp., Performance Chemicals Div., Mt. Olive, N.J.). Sulfated and phosphated derivatives of these surfactants may also be useful. Examples of such derivatives include ammonium nonoxynol-4-sulfate, which is commercially available under the trade designation RHODAPEX CO-436 from Rhodia, Dayton, N.J.

### 4. Polaxamers.

Surfactants based on block copolymers of ethylene oxide (EO) and propylene oxide (PO) may also be effective. Both EO-PO-EO blocks and PO-EO-PO blocks are expected to work well as long as the HLB is at least about 14, and preferably at least about 16. Such surfactants are commercially available under the trade designations PLURONIC and TETRONIC from BASF Corp., Performance Chemicals Div., Mt. Olive, N.J. It is noted that the PLURONIC surfactants from BASF have reported HLB values that are calculated differently than described above. In such situation, the HLB values reported by BASF should be used. For example, preferred PLURONIC surfactants are L-64 and F-127, which have HLBs of 15 and 22, respectively.

### 5. Polyalkoxylated Esters.

Polyalkoxylated glycols such as ethylene glycol, propylene glycol, glycerol, and the like may be partially or completely esterified, i.e., one or more alcohols may be esterified, with a (C₈ to C₂₂) alkyl carboxylic acid. Such polyethoxylated esters having an HLB of at least about 14, and preferably at least about 16, may be suitable for use in compositions of the present application.

### 6. Alkyl Polyglucosides.

Alkyl polyglucosides may also be used. Examples include glucopon 425, which has a (C₈ to C₁₆) alkyl chain length with an average chain length of 10.3 carbons and 1 4 glucose units.

### (d) Cationic Surfactants:

Surfactants of the cationic type that may be useful include but are not limited to primary amines, secondary amines, tertiary amines, quaternary amines, alkanolamines, mono-alkyl alkanolamines, di-alkyl alkanolamines, tri-alkyl alkanolamines, alkyl mono alkanolamines, alkyl di-alkanolamines, alkylamines, mono-alkyl amines, di-alkyl amines, tri-alkylamines, alkoxylated amines, alkyl and aryl amine alkoxylates, methoxylated alkylamines, ethoxylated alkylamines, alkoxylated alkanolamines, alkyl alkanolamines, alkoxylated ethylene diamine derivatives, alkyl/aryl/arylalkyl amine oxides. The preferred cationic surfactants of the present application would include but are not limited to (a) alkyl alkanolamines; and (b) alkyl tertiary amines. Additional information on useful cationic surfactants for the purpose of present application is well described in McCutcheon's Detergents and Emulsifiers, North American Ed., 1982 and Kirk-Othmer, Encyclopedia of Chemical Technology, 3^{rd} Ed., Vol. 22, pp. 346-387, the contents of which are included herein by reference.

If desired, combinations of various surfactants can be used for the preparation of an aqueous, stable, non-alcoholic or slightly-alcoholic antimicrobial mouthwash composition. Particularly the preferred anionic surfactants include alkyl esters of inorganic or organic acids with or without polyalkoxylated group included. These include the sulfonates, sulfates, phosphates, and phosphonates.

The presence of the water soluble polymer in the matrix facilitates formation of a polymer-surfactant complex which can lead to a micro or nano emulsion of the water-insoluble or water-immiscible antimicrobial agent even with low amounts of surfactant present in the composition. Both the use of a low level of surfactant and the complexing polymer provides a substantially irritant-free composition.

The amount of surfactant to form a micro or nano emulsion of the water-insoluble or water-immiscible antimicrobial agent in water depends on the antimicrobial agent and concentration of the material and wherein, the higher the hydrophobic material concentration, the higher the amount of surfactant to be added.

For particular compositions of the polymer-surfactant-complex and antimicrobial agent, the weight ratio of antimicrobial agents to surfactant-polymer complex may be about 1:80 to 5:0.5, preferably about 1:0.2 to 1:40. The weight ratio of antimicrobial agent to surfactant suitably is about 1:40 to 2:1, more particularly about 1:10 to 5:1, preferably about 1:8 to 1:1 and in certain embodiments about 1:5 to 1:3. The weight ratio of antimicrobial agent to polymer suitably may be about 1:10 to 5:0.5, more particularly about 1:0.2 to 1:2, preferably about 1:2 to 1:0.5. For concentrates, the antimicrobial agent will typically be present in an amount by weight of about 1% to about 40%, more particularly from about 1.5% to about 30% and in accordance with certain embodiments from about 2% to about 20% of the concentrate. Moreover, in some cases, the concentrate can also function as a use composition. The use levels of antimicrobial agents for the preparation of oral care compositions such as oral disinfecting liquids, oral disinfecting solids, oral gargling compositions, solid dosage forms, lozenges, controlled release and/or sustained release forms may be from about 0.01% wt to about 5.0% wt, more particularly 0.01% wt to about 1.0% wt. The weight ratio of the surfactant to the polymer is typically about 20:1 to 1:20, preferably about 10:1 to 1:10 and more particularly about 5:1 to 1:5.

According to one aspect of the application, a preservative or preservative system can be employed to prepare the mouthwash compositions comprising antimicrobial agents. Examples of useful preservatives or preservative systems include, but are not limited to, beeswax, d-limonene, monohydric alcohol, L-ergothioneine, ascorbic acid, sodium nitrates, sodium erythorbate, erythorbic acid, sodium succinate, grape seed extract, pine bark extract, apple extract, tea proplyphenols, succinic acid, parabens, sodium dehydro acetate, allicin, isothiocyanates, sodium benzoate, potassium sorbate, parahydroxy benzoic acid and salts thereof, (C₁-C₆) alkyl parahydroxy benzoates, sorbic acid and salts thereof, ortho hydroxybenzoic acid and salts thereof, benzoic acid and salts thereof, (C₁-C₄) alkyl benzoates, propionic acid and salts thereof, dehydroacetic acid and salts thereof, formic add and salts thereof, and undec-10-enoic acid or salts thereof alone or in combination. Preservatives or preservative systems including sodium benzoate, methyl-4-hydroxybenzoate, phenylcarbinol, benzalkonium chloride and/or thimerosal are particularly useful.

According to certain aspects, a weak carboxylic acid may be employed to prepare the mouthwash compositions, wherein it serves as an acidulant and contributes to the antibacterial activity of the composition to have improved antibacterial or antimicrobial efficacy. The weak carboxylic acid may be selected from the group including, but not limited to, tartaric acid, lactic acid, malic acid, fumaric acid, oxalic acid, benzoic acid, phosphoric acid, metaphosphoric acid, hexametaphosphoric acid, citric acid; boric acid, sulfurous acid or acetic acid alone or in combination. Suitably, this carboxylic acid should be present in the composition at a concentration of from about 0.01% to about 1.0% by weight of the total with the most desired level being about 0.1%.

The coloring agents may be used to create the preferred color. The coloring agents or colorants used in the present application include natural foods colors and dyes suitable for food, drug and cosmetic applications. These colorants are also known as F.D. & C. dyes and lakes and are preferably water-soluble in nature. Examples of representative colorants include, but are not limited to, disodium salt of 5,5-indigotindisulfonic acid (Blue No. 2), 4-[4-(N-ethyl-p-sulfoniumbenzylamino)diphenylmethylene]-[1-(N-ethyl-N-p-sulfoniumbenzyl)delta-2,5-cyclohexadien eimine] (Green No. 1), Yellow No. 10, Green No. 3 comprising a triphenylmethane dye, FD&C Blue #1, FD&C Yellow #5, FD&C Yellow #10, FD&C Red #3, FD&C Red #40; caramel color or powder (#05439), chocolate shade (#05349), green lake blend (#09236), kowet titanium dioxide (#03970), yellow liquid color (#00403), and nitrites. A full recitation of all F.D. & C. and D. & C. dyes and their corresponding chemical structures may be found in the Kirk-Othmer Encyclopedia of Chemical Technology, Volume 5, pages 857-884, which text is accordingly incorporated herein by reference. The preferred colorant of the present application is caramel color. These coloring agents may be incorporated in amount up to about 3%, more particularly up to about 2%, and in some cases less than about 1% by weight of the oral care mouthwash compositions.

In another embodiment of the present application, the antimicrobial mouthwash composition is stable for at least six months at room temperature or stable for at least about 3 freeze/thaw cycles wherein temperature cycled from 50° C. to −24° C. in every 24 hours or stable for at least 2 weeks at about 50° C.

In accordance with particular embodiments of the application, the compositions primarily comprise a polymer-surfactant complex, a hydrophobic antimicrobial agent and suitable additives. The additives may be added to the desired composition to modify or provide certain properties to the end-use compositions. Examples of such additives that can be added include, but are not limited to, flavors, colors, thickeners, defoamers, additional surfactants, antifreezing agents, pH adjusting agents, ultraviolet light stabilizers, antioxidants, co-solvents, polymers, botanical extracts, fragrances, humectants, enzymes, whitening agents, silicones, inorganic metals or salts thereof; antibacterial enhancing agents (AEA), and chlorophyll compounds.

Typically, the buffers are included in amounts that retain the pH at levels of from roughly 3.5 to about 7.0 and more preferably, from about 4.5 to 5.5. Without being bound to any theory, it is believed that these pH levels provide the essential oils with an environment that maximizes their germ killing efficacy. Buffer systems are essential to manage the pH of the composition at most favorable levels. The desired pH of the composition can be obtained by employing any suitable acidic agents such as, but not limited to, acetic acid, salicylic acid, citric acid, sulfamic acid, any weak carboxylic acids, benzoic acid or their salts, citric acid or their salts, phosphates, pyrophosphate and its salts, metaphosphate and its salts, carbonic acid and its salts, hydroxylammonium, adidic acid and its salts, maleic acid and its salts, and ascorbic acid and its salts. Practical systems include those based on sodium benzoate and benzoic acid in amounts of from approximately 0.001% w/v to about 2.0% w/v and sodium citrate and citric acid in amounts of from about 0.001% w/v to about 2.0% w/v and preferably from about 0.1% to about 0.3% w/v respectively.

In preparing oral care mouthwash composition, it is preferred to add some thickening agents to provide a desirable consistency. Examples of useful thickening agents include carboxyvinyl polymers, carrageenan, hydroxyethyl cellulose, laponite and water soluble salts of cellulose ethers such as sodium carboxymethylcellulose and sodium carboxymethyl hydroxyethyl cellulose, copolymers of lactide and glycolide monomers, carbomers. Natural gums such as gum karaya, xanthan gum, gum arabic, and gum tragacanth can also be used. Colloidal magnesium aluminum silicate or finely divided silica can be used as part of the thickening agent to further improve texture. Some thickening agents, however, except polymeric polyether compounds, e.g., polyethylene or polypropylene oxide (M.W. 300 to 1,000,000), capped with alkyl or acyl groups containing 1 to about 18 carbon atoms. Carbomers are commercially available from B.F. Goodrich as the Carbopol Series. Particularly preferred carbopols include Carbopol 934, 940, 941, 956, and mixtures thereof. Thickening agents are usually present in an amount from about 0.1% to about 15% by weight of the total toothpaste or gel composition. Higher concentrations may be used for chewing gums, lozenges and breath mints, sachets, non-abrasive gels and sub gingival gels. However, the preferred amount of thickening agent for the oral care mouthwash composition is the range of about 0.01-1.0% by weight, preferably about 0.02-0.1%.

The sweetening agent used in this application may be selected from a wide range of agents, including natural, artificial and water-soluble sweeteners, water-soluble artificial sweeteners, and/or dipeptide based sweeteners. The representative illustrations encompass water-soluble sweetener such as monosaccharides, disaccharides, and polysaccharides such as xylose, ribose, glucose, mannose, galactose, fructose, dextrose, sucrose, maltose, partially hydrolyzed starch or corn syrup solids and sugar alcohols such as sorbitol, xylitol, mannitol, hydrogenated glucose syrup and mixtures thereof; and water-soluble artificial sweeteners such as the soluble saccharin salts, i.e., sodium or calcium saccharin salts, cyclamate salts, such as the sodium salt and the like, and the free acid form of saccharin; dipeptide based sweetening agents such as L-aspartyl-L-phenyl-alanine methyl ester and materials; dihydrochalcone; glycyrrhizin; Stevia rebaudiana (Stevioside); and the synthetic sweetener 3,6-dihydro-6-methyl-1,2,3-oxathiazin-4-one-2,2-dioxide, particularly the potassium (Acesulfame-K), sodium and calcium salts thereof. The amount of the sweetener will vary with the type of sweetener selected and the desired level of sweetness. Water-soluble sweeteners derived from naturally occurring water-soluble sweeteners, such as a chlorinated derivative of sucrose is known under the product description of sucralose as well as protein based sweeteners such as thaumatococus danielli (Thaumatin I and II) can be used, Sweetening agents are typically used in oral care compositions at levels of from about 0.005% to about 5%, by weight of the composition.

Another constituent of the present oral care mouthwash compositions desired herein is a humectant, and it serves to keep the compositions from hardening upon contact to air, and certain humectants can also contribute desirable sweetness of flavor to the compositions. The additional ingredient which assists solubilization of the antibacterial or antimicrobial agent in saliva may be incorporated in the water-humectant vehicle. Such agents include glycerin, sorbitol, polyethylene glycol, propylene glycol, other edible polyhydric alcohols, poloxamers, and combination thereof. Polyols such as propylene glycol, dipropylene glycol, and hexylene glycol, cellosolves such as methyl cellosolve and ethyl cellosolve, vegetable oils and waxes containing at least about 12 carbons in a straight chain such as olive oil, castor oil and petrolatum and esters such as amyl acetate, ethyl acetate and benzyl benzoate. As used herein “propylene glycol” includes 1,2-propylene glycol and 1,3-propylene glycol. The total amount of humectant generally comprises from about 0.5% to 20%, preferably from about 1% to 10% by weight of the compositions herein.

Optionally, about 0.1 wt % to about 5.0 wt % of antifoaming or defoamers may be employed to stop any unwanted foam generated while manufacturing the antimicrobial oral care mouthwash composition of the present application. Examples of antifoaming agents include silicone based compounds, alcohols, glycol ethers, mineral spirits, acetylene diols, polysiloxanes, organosiloxanes, siloxane glycols, reaction products of silicon dioxide and organosiloxane polymer, polydimethylsiloxanes or polyalkylene glycols alone or in combination.

The mouthwash composition optionally includes at least one activating agent such as peroxide stabilized peroxide, fluoride and/or alkali salts.

The peroxide or stabilized peroxide compound enhances the efficacy of the present composition by causing pore channel formation and/or delipidization of bacterial membranes. It is further hypothesized that (i) the peroxide may boost the anti-plaque efficacy of the composition through hydrogen bonding, superoxide formation and/or a synergistic antimetabolic affect with the associated antimicrobial constituents; (ii) increased peroxide stress in the saliva and plaque; and (iii) an increased diffusion of the antimicrobial agents into the oral cavity. A variety of peroxygen compounds that can be employed include, but are not limited to, urea peroxide, calcium peroxide, hydrogen peroxide and the salts of perborate, persilicate, perphosphate and percarbonate. The most appropriate peroxide compound for this application is hydrogen peroxide in the range of from about 0.1 to about 10% by weight.

Another important activating agent is a source of fluoride ions or a fluorine providing element as an anti-caries agent in an amount sufficient to deliver from about 25 ppm to 5000 ppm of fluoride ions. The preferred fluoride source for the present application may be selected from the group including, but not limited to, inorganic fluoride salts, e.g., soluble alkali metal, alkaline earth metal salts such as sodium fluoride, potassium fluoride, ammonium fluoride, calcium fluoride, copper fluoride, cuprous fluoride, zinc fluoride, barium fluoride, sodium fluorosilicate, ammonium fluorosilicate, sodium fluorozirconate, ammonium fluorozirconate, sodium monofluorophosphate, aluminum mono and/or di-fluorophosphate, fluorinated sodium calcium pyrophosphate, alkali metals, tin fluorides, sodium fluorides, stannous fluorides, sodium monofluorophosphate alone or in combination.

In accordance with one aspect of the present application, there is provided a process for preparing an aqueous, stable, non-alcoholic or slightly-alcoholic antibacterial mouthwash composition with an improved antimicrobial efficacy comprising the steps of (i) preparing a mixture of (a) a water-soluble matrix composite and (b) essential oil based antibacterial agents; (ii) dissolving the mixture of step (i) in an aqueous medium, optionally comprising an effective amount of alcohol to yield a clear microemulsion; and (iii) incorporating preservative, weak carboxylic acid, coloring agent and additives, if any, to the microemulsion of step (ii) and vigorously mixing the composition to provide a stable, homogenous, optically clear mouthwash composition.

According to one embodiment of the application, an aqueous, stable, non-alcoholic microemulsion based antibacterial mouthwash composition comprising (i) a water-soluble matrix composite made from (a) about 0.10% wt of polyvinyl pyrrolidone (PVP); and (b) about 0.5% wt of sodium lauryl sulfate (SLS); (ii) a mixture of (a) about 0.064% wt of thymol; (b) about 0.042% wt of menthol; (c) about 0.092% wt of eucalyptol; and (d) about 0.060% wt of methyl salicylate; (iii) about 0.15% wt of sodium benzoate; (iv) about 0.15% wt of benzoic acid; and (v) about 1.0% wt of natural caramel is provided.

Another embodiment of the application is directed to an aqueous, stable, slightly-alcoholic microemulsion based antibacterial mouthwash composition comprising (i) a water-soluble matrix composite made from (a) about 0.10% wt of polyvinyl pyrrolidone (PVP) and (b) about 0.5% wt of sodium lauryl sulfate (SLS); (ii) a mixture of (a) about 0.064% wt of thymol; (b) about 0.042% wt of menthol; (c) about 0.092% wt of eucalyptol; and (d) about 0.060% wt of methyl salicylate; (iii) about 0.15% wt of sodium benzoate; (iv) about 0.15% wt of benzoic acid; (v) about 1.0% wt of natural caramel; and (vi) about 3.0% wt to about 10% wt of alcohol based on the total composition.

In order to prepare the aqueous non-alcoholic antimicrobial mouthwash compositions, the selected aqueous medium can be any type of water that is known in the art for this purpose and must comply with the standards of United States Pharmacopoeia (USP), preferably selected from distilled water, de-ionized water, double distilled water, triple distilled water, tap water, de-mineralized water, reverse-osmosis water alone or in combination.

The levels of alcohol employed to prepare the slightly-alcoholic microemulsion based antibacterial mouthwash composition may be in the range of about 1% wt to about 10% wt.

In accordance with the present application, the mouthwash composition water-soluble matrix composite comprising a water-insoluble or water-immiscible antimicrobial agent is capable of inhibiting or killing oral microflora including, but not limited to, Actinomyces viscosus, alpha Streptococcus, Candida albicans, Escherichia coli, Pseudomonas aeruginosa, Staphylococcus epidermidis, Streptococcus nutans, Streptococcus Bovis, Streptococcus salivarius, Fusobacterium nucleatum, Prevotella melaninoaenica, Streptococcus sanguis, Campylobacter rectus, Fusobacterium nucleatum, Porphyromonas gingivalis, Staphyloccus aureus, Staphyloccus albus, Streptococcus viridians, Streptococcus hermolyticus, Neisseria catarrhalis, Diplococcus pneumonia, Klebsiella pneumonia, Coronebacterium diptheriae, Mycobacterium phlei, Eberthella typhosa, Proteus vulgaris, Shigella dysenteriae, Shigella paradynsenteriae, Shigella paraidysenteriae, Shigella sonne, and/or Lactobacillus casei.

The effective amount of the antimicrobial mouthwash compositions of the present application may be used by bringing the compositions into contact with (a) mucosal or gingival tissue of the oral cavity, and/or (b) the surface of the teeth for prophylactic or therapeutic purposes of the user with regard to diseases or conditions of the oral cavity. The mouthwash compositions of the present application can be formulated as solid, paste, powder, dental tablet, dental gel, solution, gum, lozenge, mouth rinses, liquid dentifrices, dental films, dental strips, paint on gels, dental beads, confectionaries, toothpastes and dental cream treat bad breath, calculus, caries, plaque formation and the like. Various forms of these formulations can be supplied in deformable tubes, pump dispensers, pressurized dispensers, packets, bottles, jars, aluminium strips, and other suitable containers.

In accordance with a particularly useful embodiment, a non-alcoholic flavor base concentrate is manufactured comprising a mixture of (a) a water-soluble matrix composite made of water-soluble polymer and water-soluble surfactant; (b) flavors including thymol, menthol, eucalyptol and methylsalicylate; and (c) water. The flavor base concentrate can be further diluted to produce antimicrobial mouthwash composition with or without alcoholic content and wherein the preferred level of alcohol is in the range of about 0% wt/v to about 10% wt/v of the composition. The specific compositions of flavor base concentrate, non-alcoholic or slightly alcoholic diluted mouthwash compositions are provided in examples 1, 4, 5 and 6 of this specification.

Example 3 represents an alcohol-free mouthwash composition based on example 1 of U.S. Pat. No. 5,817,295 (“Alcohol Free Mouthwash” assigned to Warner-Lambert Company). The composition is described therein as being capable of preventing and eliminating bad breath and killing and/or inhibiting the oral microflora that are responsible for plaque formation. For the purpose of establishing an alcohol free control composition, the example 1 of this patent is modified in such a way to match the components of a commercially available amber-color thymol based mouthwash composition excluding its alcoholic part. This composition served as a “control” for the comparison of antimicrobial efficacy of non-alcoholic and slightly-alcoholic compositions of the present invention.

Further, the present invention is illustrated in detail by way of the following examples. The examples are given herein for illustration of the invention and are not intended to be limiting thereof.

### Example 1

### Flavor Base Concentrate

| Component | Weight % |
|---|---|
| Sodium Lauryl Sulfate | 15.00 |
| Plasdone K-29/32 | 3.40 |
| Thymol | 2.56 |
| Menthol | 1.68 |
| Methyl Salicylate | 2.40 |
| Eucalyptol | 3.68 |
| Water | 71.28 |
| Total | 100.00 |

### Example 2 (Comparative)

### Lab Prepared Commercially Available Amber-Color Thymol Based Mouthwash Composition

| Component | Weight % |
|---|---|
| Water | 71.867 |
| Ethanol | 26.900 |
| Pluronic F127 | 0.075 |
| Benzoic Acid | 0.150 |
| Sodium Benzoate | 0.150 |
| Eucalyptol | 0.092 |
| Thymol | 0.064 |
| Methyl Salicylate | 0.060 |
| 1-Menthol | 0.042 |
| Caramel Color (1%) | 0.600 |
| Total | 100.000 |

### Example 3 (Comparative)

### Based on Example 1 of U.S. Pat. No. 5,817,295

| Component | Weight % |
|---|---|
| Water | 90.132 |
| Glycerin | 7.500 |
| Sodium Lauryl Sulfate | 0.210 |
| Pluronic F127 | 0.500 |
| PEG 600 | 0.500 |
| Benzoic Acid | 0.150 |
| Sodium Benzoate | 0.150 |
| Eucalyptol | 0.092 |
| Thymol | 0.064 |
| Methyl Salicylate | 0.060 |
| 1-Menthol | 0.042 |
| Caramel Color (1%) | 0.600 |
| Total | 100.00 |

### Example 4

### Alcohol Free Mouthwash Composition Prepared Employing Flavor Base Concentrate of Example 1

| Component | Weight % |
|---|---|
| Water | 96.600 |
| Flavor Base of example 1) | 2.500 |
| Benzoic Acid | 0.150 |
| Sodium Benzoate | 0.150 |
| Caramel Color (1%) | 0.600 |
| Total | 100.000 |

### Example 5

### Reduced Alcohol Mouthwash Composition Prepared Employing Flavor Base Concentrate of Example 1

| Component | Weight % |
|---|---|
| Water | 94.600 |
| Flavor Base (example 1) | 2.500 |
| Benzoic Acid | 0.150 |
| Sodium Benzoate | 0.150 |
| Caramel Color (1%) | 0.600 |
| Ethanol (Absolute) | 2.000 |
| Total | 100.00 |

### Example 6

### Reduced Alcohol Composition Prepared Employing Flavor Base Concentrate of Example 1

| Component | Weight % |
|---|---|
| Water | 86.60 |
| Flavor Base (example 1) | 2.50 |
| Benzoic Acid | 0.15 |
| Sodium Benzoate | 0.15 |
| Caramel Color (1%) | 0.60 |
| Ethanol (Absolute) | 10.00 |
| Total | 100.00 |

### Example 7

### Cold and Elevated Temperature Stability Studies

The stability study is conducted on flavor base concentrate of Example 1 and alcohol free mouthwash composition of Example 4. Small amounts of product are placed in a storage shelf at ambient (lab) temperature, −15° C. (3 cycles freeze/thaw) and 50° C. Each sample is placed in a freezer at −15° C. and allowed to freeze until it becomes solid. The samples are removed and allowed to thaw at ambient temperature. No phase separation or stratification is observed. This process is repeated two more times with identical results. Samples of the above compositions are also placed in a 50° C. oven and allowed to stand for approximately 10 days and found to be stable without any signs of phase separation, stratification or discoloration. Further, all the compositions of example 1 through 6 are kept in 40° F. for four months and they are found to be stable.

### Example 8

### Determination of Antimicrobial Efficacy

Minimum Inhibitory Concentration study (MICs) is performed on all five different mouthwash compositions of examples 2 to 6. The microbial strains are chosen according to Balm kill kinetic studies of U.S. Pat. No. 5,817,295. The Balm study is conducted to determine the time it takes to kill specific microbial strains. Table I reveals a summary of the results of the MIC studies. Static (stops growth) and Cidal (kills all organisms) minimum concentrations of test product (in ppm) are shown for each microorganism. It is observed that the compositions of examples 4, 5 and 6 (0%, 2% and 10% ethanol respectively) tendered better antimicrobial activity against S. Mutans (causes dental caries) and A. Viscosis (implicated in gingivitis) than the mouthwash compositions of example 2 and example 3. Adding ethanol to the composition of example 4 does not improve the antimicrobial performance. All five compositions (examples 2 to 6) showed similar results with respect to C. albicans (a cause of Thrush). Example 3 is not effective against P. Aeruginosa (biofilms/dental plaques) at the tested level; however, the performance of the remaining four formulations against P. Aeruginosa organism is significantly comparable.

TABLE I
Minimum Inhibitory Concentration studies (in ppm)

| Compo- | S. mutants | | A. viscosus | | P. aeruginosa | | C. albicans | |
|---|---|---|---|---|---|---|---|---|
| sitions | Static | Cidal | Static | Cidal | Static | Cidal | Static | Cidal |
| Example 2 | 80 | 320 | 40 | 160 | 80 | 160 | 80 | 320 |
| Example 3 | 20 | 320 | 20 | 40 | >320 | N/A | 80 | >320 |
| Example 4 | 10 | 80 | 10 | 40 | 80 | >320 | 40 | 160 |
| (0% ethanol) | | | | | | | | |
| Example 5 | 10 | 160 | 10 | 40 | 80 | >320 | 40 | 320 |
| (2% ethanol) | | | | | | | | |
| Example 6 | 10 | 80 | 10 | 20 | 80 | 320 | 40 | 320 |
| (10% | | | | | | | | |
| ethanol) | | | | | | | | |

### Example 9

### Determination of Active Ingredients in Various Compositions Through High Performance Liquid Chromatography (HPLC)

An HPLC analysis of the active ingredients in the lab prepared commercially available amber-color thymol based mouthwash composition (example 2) is performed in order to confirm that every active ingredient is present in the same amount or concentration in each one of the five compositions. Every sample is evaluated in duplicate at both 1:1 and 1:9 dilutions in acetonitrile. Results of this analysis are provided in Table II along with the calculated and theoretical quantities of each active ingredient added in respective compositions. In general, the measured 1:1 dilutions are somewhat less than the calculated theoretical amounts. However, the 1:9 dilutions are all very close in value for each active ingredient and are very close to theoretical values with the exception of menthol which is much higher than theoretical.

TABLE II
HPLC determination of essential oils in antimicrobial composition

| | Methyl | | | |
|---|---|---|---|---|
| Compositions | salicylate | Thymol | Eucalyptol | Menthol |
| | 1:1 (Product:Acetonitrile) | | | |
| Example 2 | 285 | 281 | 381 | 579 |
| Example 3 | 235 | 226 | 354 | 470 |
| Example 4 (0% ethanol) | 288 | 290 | 447 | 648 |
| Example 5 (2% ethanol) | 283 | 289 | 429 | 597 |
| Example 6 (10% ethanol) | 287 | 288 | 417 | 591 |
| Commercially available | 304 | 283 | 451 | 575 |
| amber-color thymol based | | | | |
| mouthwash solution | | | | |
| Theoretical | 300 | 320 | 460 | 210 |
| | 1:9 (Product:Acetonitrile) | | | |
| Example 2 | 53 | 53 | 68 | 108 |
| Example 3 | 51 | 54 | 60 | 118 |
| Example 4 (0% ethanol) | 50 | 53 | 69 | 111 |
| Example 5 (2% ethanol) | 52 | 54 | 65 | 106 |
| Example 6 (10% ethanol) | 52 | 54 | 75 | 110 |
| Commercially available | 57 | 53 | 77 | 119 |
| amber-color thymol based | | | | |
| mouthwash solution | | | | |
| Theoretical | 60 | 64 | 92 | 42 |

While this invention has been described in detail with reference to certain preferred embodiments, it should be appreciated that the present application is not limited to those precise embodiments. Rather, in view of the present disclosure, which describes the current best mode for practicing the invention, many modifications and variations would present themselves to those skilled in the art without departing from the scope and spirit of this invention.

What is claimed is:

1. An aqueous, stable, non-alcoholic or slightly-alcoholic microemulsion based antibacterial mouthwash composition with improved antimicrobial efficacy comprising:
i. a water-soluble matrix composite made of (a) a water-soluble polymer, and (b) a water-soluble surfactant;
ii. at least one water-immiscible or water-insoluble antibacterial agent; and
iii. optionally, a preservative or preservative system, a weak carboxylic acid, a coloring agent and other additives.

2. The mouthwash composition according to claim 1, wherein the composition is stable for at least six months at room temperature or stable for at least 3 freeze/thaw cycles wherein the temperature is cycled from 50° C. to −24° C. in every 24 hours or stable for at least 2 weeks at about 50° C.

3. The mouthwash composition according to claim 1, wherein the slightly-alcoholic composition comprises about 0.1% wt to about 20% wt alcohol based on the total composition.

4. The mouthwash composition according to claim 1, wherein the water-soluble matrix is a complex of (a) and (b) having a lower critical micelle concentration (cmc) than a composition without the water-soluble polymer.

5. The mouthwash composition according to claim 1, wherein said water-soluble polymer is selected from the group consisting of acids, esters, amides or salts of olefinic polymers, lactam/pyrrolidone based polymers, pyrrolidone co-polymers, α-olefin maleic acid/ester co-polymers, α-olefin polymers, carbohydrate based polymers, natural polymers, natural gums and combinations thereof.

6. The mouthwash composition according to claim 1, wherein said water-soluble surfactant is selected from the group consisting of anionic, non-ionic, amphoteric, cationic surfactants and combinations thereof.

7. The mouthwash composition according to claim 6, wherein said water-soluble surfactant is selected from the group consisting of sulfonates, sulfates, phosphates, phosphonates, amine oxides, ammonium carboxylates, ammonium sulfonates, polysorbates, polyalkoxylated alkanols, polyalkoxylated alkylphenols, polyalkoxylated esters, EO/PO copolymers, poloxamers, alkyl polyglucosides, naturally occurring surface active compositions, phospholipids, fatty acid based surfactants, surface active homo or copolymers of polyamines, polyimines, polyalkyleneimines, alkyl/aryl amine alkoxylates, alkyl/aryl/arylalkyl amine oxides, alkoxylated ethylene diamine derivatives, and combinations thereof.

8. The mouthwash composition according claim 1, wherein said water insoluble antibacterial agent is an essential oil based antibacterial agent and is selected from the group consisting of triclosan, thymol, eucalyptol, menthol, methyl salicylate, carvacrol, limonene, terpinene, phellandrene, pinene, citral, methylheptenone, citronellal, geraniol, linalool, β-pinene, limonene, anethole, safrol, eugenol, eugenyl acetate, caryophyllene, citronellol, borneol, bornyl esters, camphor, geraniol, linalyl acetate, camphene, menthyl esters, carvone, pinene, safrole, acetaldehyde, camphor, myrcene, chavicol, cinnamaldehyde, cinnamyl acetate, terpinen-4-ol, cineole, α-thujone, β-thujone, fenchone, natural available disinfecting plant products/extracts like neem, turmeric, and combinations thereof.

9. The mouthwash composition according claim 1, wherein said preservative is selected from the group consisting of sodium benzoate, methyl-4-hydroxybenzoate, phenylcarbinol, benzalkonium chloride, thimerosal, and combinations thereof.

10. The mouthwash composition according claim 1, wherein said composition further comprises an additive or activating agent selected from the group consisting of stabilized peroxide, fluorides, alkali salts, and combinations thereof.

11. The mouthwash composition according claim 1, wherein said composition comprises a weak carboxylic acid selected from the group consisting of citric acid, tartaric acid, lactic acid, benzoic acid, and combinations thereof.

12. The mouthwash composition according claim 1, wherein said composition comprises a coloring agent selected from F, D & C dyes, naturally-derived colors, and combinations thereof.

13. The mouthwash composition according claim 1, wherein the composition is optically clear.

14. The mouthwash composition according claim 1, wherein the weight ratio of antibacterial agent to water-soluble polymer is about 1:10 to 5:0.5.

15. The mouthwash composition according claim 1, wherein the weight ratio of antibacterial agent to water-soluble polymer is about 1:2 to 1:0.5.

16. The mouthwash composition according claim 1, wherein the weight ratio of antibacterial agent to water-soluble surfactant is about 1:10 to 5:1.

17. The mouthwash composition according claim 1, wherein the weight ratio of antibacterial agent to water-soluble surfactant is about 1:3 to 2:1.

18. The mouthwash composition according claim 1, wherein the weight ratio of (a):(b) is about 20:1 to 1:20.

19. The mouthwash composition according claim 1, wherein the weight ratio of (a):(b) is about 5:1 to 1:5.

20. The mouthwash composition according claim 1, wherein said antibacterial agent is present in an amount of about 10 ppm to about 20% by weight of the composition.

21. The mouthwash composition according claim 1, wherein the composition is capable of inhibiting or killing gram (−) and/or gram (+) bacterial strains.

22. The mouthwash composition according claim 1, wherein said composition comprises an additive selected from the group consisting of flavors, thickeners, defoamers, buffers, sweeteners, humectants, softeners, astringents, and combinations thereof.

23. The mouthwash composition according claim 1, wherein said water-soluble polymer comprises polyvinyl pyrrolidone (PVP) in the range of about 0.01% wt to about 1% wt based on the total composition.

24. The mouthwash composition according claim 1, wherein said water-soluble surfactant comprises sodium lauryl sulfate in the range of about 0.1% wt to 5.0%.

25. The mouthwash composition according claim 24, wherein said sodium lauryl sulfate is in the range of about 2% wt of the total composition.

26. The mouthwash composition according claim 1, wherein said water insoluble antibacterial agent comprises thymol in the range of about 0.01% wt to about 1.0% wt of the total composition.

27. The mouthwash composition according claim 1, wherein said water insoluble antibacterial agent comprises menthol in the range of about 0.01% wt to about 1.0% wt of the total composition.

28. The mouthwash composition according claim 1, wherein said water insoluble antibacterial agent comprises eucalyptol in the range of about 0.01% wt to about 1.0% wt of the total composition.

29. The mouthwash composition according claim 1, wherein said water insoluble antibacterial agent comprises methyl salicylate in the range of about 0.01% wt to about 1.0% wt of the total composition.

30. The mouthwash composition according to claim 1 in the form of an oral disinfecting liquid, oral disinfecting solid, oral gargling composition, solid dosage forms, controlled release forms and/or sustained release forms.

31. The mouthwash composition according to claim 30, wherein said composition is in the form of an oral disinfecting solid dosage form which is an effervescent matrix with a sodium bicarbonate solid base and citric acid.

32. A process for preparing an aqueous, stable, non-alcoholic or slightly-alcoholic microemulsion based antibacterial mouthwash composition comprising the steps of:
i. preparing a mixture of (a) a water-soluble matrix composite and (b) an essential oil based antibacterial agent;
ii. dissolving the mixture of step (i) in an aqueous medium, optionally comprising an effective amount of alcohol to yield a clear microemulsion; and
iii. optionally incorporating a preservative, weak carboxylic acid, coloring agent and/or other additives to the microemulsion of step (ii) and vigorously mixing the composition to result in a stable, homogenous, optically clear mouthwash composition.

33. An aqueous, stable, non-alcoholic microemulsion based antibacterial mouthwash composition comprising:
i. a water-soluble complex made from (a) about 0.10% wt of polyvinyl pyrrolidone (PVP); and (b) about 0.5% wt of sodium lauryl sulfate (SLS);
ii. a mixture of (a) about 0.064% wt of thymol; (b) about 0.042% wt of menthol; (c) about 0.092% wt of eucalyptol; and (d) about 0.060% wt of methyl salicylate;
iii. about 0.15% wt of sodium benzoate;
iv. about 0.15% wt of benzoic acid; and
v. about 1.0% wt of natural caramel.

34. An aqueous, stable, slightly-alcoholic microemulsion based antibacterial mouthwash composition comprising:
i. a water-soluble complex made from (a) about 0.10% wt of polyvinyl pyrrolidone (PVP) and (b) about 0.5% wt of sodium lauryl sulfate (SLS);
ii. a mixture of (a) about 0.064% wt of thymol; (b) about 0.042% wt of menthol; (c) about 0.092% wt of eucalyptol; and (d) about 0.060% wt of methyl salicylate;
iii. about 0.15% wt of sodium benzoate;
iv. about 0.15% wt of benzoic acid;
v. about 1.0% wt of natural caramel; and
vi. about 3.0% wt to about 10% wt of alcohol based on the total composition.
